(19) United States
(12) Patent Application Publication
(10) Pub. No.: US 2019/0038468 A1
(43) Pub. Date: Feb. 7, 2019

(54) A PROTECTIVE EYEWEAR DEVICE

(71) Applicant: EC MEDICA LTD, Southport Merseyside (GB)

(72) Inventor: Paul BENNETTS, Southport (GB)

(21) Appl. No.: 16/061,341

(22) PCT Filed: Oct. 19, 2016
(86) PCT No.: PCT/GB2016/053264

§ 371 Date: Jun. 11, 2018

(30) Foreign Application Priority Data
Oct. 20, 2015 (GB) 1518590.3

(51) Int. Cl. A61F 9/04

(52) U.S. Cl. CPC A61F 9/04

(57) ABSTRACT

A protective eyewear device comprising a deformable main body portion, the main body portion comprising one or more ocular apertures; at least one cut-out intersecting an edge of the main body portion adapted, in use, to conform to the shape of the bridge of a wearer's nose; a protective layer affixed to a first surface of the main body; and an adhesive layer affixed to a second surface of the main body portion opposite the first surface. Suitably, the protective layer is continuous (i.e. does not have any holes in it), and covers the entire first surface of the main body. Suitably, the protective layer comprises a thin, flat sheet of plastics material.

### CROSS-REFERENCE TO RELATED APPLICATIONS

This application is a US national phase application out of PCT/GB2016/053264, filed on 19 Oct. 2016 and published as WO2017068346 on 27 Apr. 2017. This application claims the benefit and priority of UK patent application number GB1518590.3, filed on 20 Oct. 2015. The entire disclosure of each above-cited application is hereby incorporated herein by reference.

### BACKGROUND

This invention relates to a protective eyewear device, and in particular, but without limitation to, a protective eyewear device suitable for use during operative procedures (i.e. in theatre), and/or in day clinics where certain procedures are carried out that require eye protection.

During operative or other similar procedures, one of the primary concerns of the operating practitioner is the welfare of the patient. Of high importance when considering the patient's welfare is ensuring that the patient's eyes are not vulnerable to injury during the procedure, such as: drying out of the cornea; accidental contact with blood, other liquids and debris; corneal abrasions; and haemorrhages of the eyeball.

Conventionally, during the procedure, the patient's eyes are taped closed using surgical tape, thus preventing debris from entering the eye. However, this method may not effectively prevent blood and/or fluids from entering the eye. Furthermore, it is occasionally preferable to be able to monitor the patient's pupils when under anaesthetic, which would of course not be achievable if the eyes are taped closed.

Protective eyewear devices suitable for use in surgery are already available, which provide adequate protection for the eye, whilst also permitting the practitioner to see the patient's eyes during a procedure. Examples of known protective eyewear devices not in accordance with the present invention are described the following published patent and utility model applications: WO 2016/029257A1 [WALLACE COOMBE PTY]; US 2002/100481A1 [QAMAR ABBASI]; DE 202015003823U [TUNC]; U.S. Pat. No. 4,788,847A [CRAIG]; and U.S. Pat. No. 7,748,387B1 [VU & JORDAN].

Nevertheless, a schematic illustration of a known protective eyewear device 10, suitable for use in surgery, is shown in FIGS. 1 and 2 of the drawings.

Referring to FIGS. 1 and 2, the protective eyewear device 10 comprises a main body portion 12 manufactured from a strip of foam cut to the desired shape: having a nose cut-out 14 and a pair of spaced-apart eye cut-outs 16. The eye cut-outs 16 are each covered by a vacuum-formed blister 18 made from transparent plastic, which each form hollow “capsules” that cover, and protect, the eyes. The blisters 18 have peripheral flanges 20 that are sandwiched between the back side of the main body portion 12 and a self-adhesive layer 22. The blisters 18 comprise side walls 24 that protrude upwardly through the eye cut-outs 16 and a generally flat lens portion 26 that protect the patient's eyes. The known protective eyewear device is placed onto a patient's face, and stuck there using the self-adhesive layer 22 (initially covered by a peel-off layer, not shown) so that the blisters 18 register with, and cover, both of the patient's eyes.

Although known devices of this general type provide a solution to the aforementioned problem of providing protection for the eyes whilst permitting them to be viewed, they tend to be associated with a number of drawbacks, as follows:

Firstly, the known device has two separate vacuum-formed blisters 18 which need to be moulded and fixed into the main body 12. In order to achieve this, the capsules 18 are formed with peripheral lips 20 that are captured between the foam 12 and the adhesive layer 22. This means that the manufacturing process of the known device 10 is relatively laborious and therefore expensive. Furthermore, polyethylene terephthalate (PET) is generally used to manufacture the blisters 18, which is a relatively expensive material. Unfortunately, healthcare providers, particularly in the UK, are increasingly constrained by budgets, and as such a more affordable, disposable device would be advantageous.

Secondly, the nose cut-out 14 provided on the known devices 10 are somewhat of a “one-size fits all” nature. This means that a “one size fits all” device 10 may not correctly fit different patient populations with different anatomical configurations. The solution to this is to provide a range of protectors 10, which disadvantageously increases the hospital's inventory. Moreover, if the wrong-sized protector 10 is used on a patient, it could be uncomfortable and, more importantly, may not be effective, as it could fit loosely (thus permitting foreign object ingress); or it could pinch the patient's skin (leading to contusions and/or pressure sores).

Thirdly, due to the relatively high cost of the “capsules” of the known eye protectors, they tend to be made as small as possible. This results in the self-adhesive portion of the eye protector sticking to the patient's eyebrows, which prevents a seal from being formed, and also can result in removal of eyebrow hair upon peeling the device off the patient's face after use.

Fourthly, known devices, such as those disclosed in WO2016/029257A1, have an aperture protective layer. The hole(s)/aperture(s) of known goggles can be provided for various reasons, such as to form a hinge and/or vent holes to prevent condensation, but this means that the protective layer is discontinuous. As such, known devices do not adequately protect the wearer's eyes from splashes and nor are they wipeable because wiping the goggles dampens the underlying foam layer and/or exposes the wearer's eyes to cleaning solvents.

A need therefore exists for an alternative and/or an improved protective eyewear device, which addresses or overcomes one or more of the above problems.

### SUMMARY

Various aspects of the invention are set forth in the appendent claims.

According to an aspect of the invention, there is provided a protective eyewear device comprising a deformable main body portion, the main body portion comprising one or more ocular apertures; at least one cut-out intersecting an edge of the main body portion adapted, in use, to conform to the shape of the bridge of a wearer's nose; a protective layer affixed to a first surface of the main body; and an adhesive layer affixed to a second surface of the main body portion opposite the first surface.

According to another aspect of the invention, there is provided a protective eyewear device comprising a deformable main body portion, the main body portion comprising one or more ocular apertures; at least one cut-out intersecting an edge of the main body portion adapted, in use, to conform to the shape of the bridge of a wearer's nose; a protective layer affixed to, and covering the entire surface of, a first surface of the main body; and an adhesive layer affixed to a second surface of the main body portion opposite the first surface.

Suitably, the protective layer is continuous, which means that it does not comprise any apertures, holes or perforations.

Suitably, the main body is resiliently deformable. The main body portion may be manufactured from a sheet of foam, such as any one or more of: an open-cell polymer foam; a closed-cell polymer foam; an open-cell elastomer foam; a closed-cell elastomer foam; an open-cell memory foam; and a closed-cell memory foam. Closed-cell foams are preferred because they inhibit or prevent the absorption and/or transduction of liquids into or through the main body of the device. Preferably, the main body portion is deformable so that it can bend to allow it to conform to the profile of a wearer's face, in use.

The main body portion comprises one or more ocular apertures. In one embodiment of the invention, there is a generally slot-shaped ocular aperture whose periphery is selected to surround the eyes of a patient, in use. In another embodiment of the invention, there are two ocular apertures, each ocular aperture having a periphery that is shaped and dimensioned to surround an eye of a patient, in use, and which ocular apertures are spaced-apart so as, in use, to register with the patient's eyes. In certain embodiments of the invention, the main body portion of the protector may be of a generally figure-of-eight shape thus forming a pair of spaced-apart ocular apertures.

Suitably, the ocular aperture, or apertures, are shaped and sized to correspond with the eyes of the wearer, such that the wearer's eyes are not obstructed, and/or visible when wearing the protector. Certain treatments may require the eye to be maintained in a dry condition rather than moist, which the eye protectors of the invention will also do.

The main body has at least one cut-out (i.e. a nose cut-out) that intersects an edge of the main body portion, which cut-out is adapted, in use, to conform to the shape of the bridge of a wearer's nose.

Suitably, there are two, differently-shaped and/or differently-sized nose cut-outs. Suitably, the nose cut-outs are positioned on opposite edges of the main body portion, which configuration may enable a single device to fit two different populations of patient, for example by inverting the main body portion, as appropriate.

A further benefit of having nose cut-outs located on opposite edges of the main body portion is that the external shape of the device thus provides a cut-out located above the bridge of the nose, near to, or on the forehead. This can provide an unobstructed portion of the face located above the eye protector upon which an electrode or needle can be placed in contact with the skin. Such an electrode or needle may be used in neuro navigation procedures involving monitoring of evoked potentials to load the cranial nerves. These systems, in fact, provide for the possibility of being able to use the cutaneous surfaces of the face of the patient in order to be able to position magnetic sensors or needles for the uptake of bioelectric stimuli. The particular shape of the eye protector of the invention allows this to work safely without having to remove the protector even in the case of placement of these surgical devices.

The nose cut-outs are suitably provided on the upper and lower horizontal edges of the main body portion. The nose cut-outs provide a secure fit, which may prevent the device from moving during a procedure.

The main body portion is suitable shaped and dimensioned so that it extends laterally beyond both of the wearer's eyes, terminating at, or near, the temples. By manufacturing the main body portion from a deformable material, these criteria can be met by the appropriate selection of dimensions and elasticity of the material from which the main body portion is manufactured.

The adhesive layer of the protector may comprise a self-adhesive layer, and preferably comprises a pressure sensitive self-adhesive. This may sealingly affix the device to the wearer's face following pressure being applied to the exterior surface of the main body portion.

Suitably, the adhesive layer may cover the entire inner surface area of the device, which may permit the entire inner surface, in particular around its periphery to be (sealingly) affixed to the wearer's face. Thus, the ingress of debris from any position around the periphery of the device may be inhibited or avoided.

The protective layer is suitably translucent or transparent, the latter being preferred as it permits, in use, the patient's eyes to be seen. The protective layer of the protector is suitably manufactured from a flexible material, for example an acrylic or acetate material, and is configured, in use, to provide a physical barrier between the environment and the wearer. In a preferred embodiment of the invention, the protective layer covers the entire first surface area of the main body. By covering the entire first (exterior) surface of the main body, the protective cover may provide a wipeable barrier between the environment and the main body portion. Preferably, the protective cover layer is substantially flat. This may allow the cover to be cleaned easily during procedures. In certain respects, a preferred embodiment of the invention differs from known surgical eye protectors insofar as the protective layer is provided as a flat sheet of material (as opposed to being vacuum-formed, or comprising profiled capsules) that overlies the entire first surface (as opposed to having peripheral lips that are interposed between the foam and adhesive layers).

In an embodiment of the invention, the device comprises a laminated structure, comprising: a first protective layer, an interposed foam layer, and an adhesive layer.

### BRIEF DESCRIPTION OF THE DRAWINGS

Preferred embodiments of the invention shall now be described, by way of example only, with reference to the accompanying drawings, in which:

FIG. 1 is a perspective view of a known eye protector;

FIG. 2 is a schematic cross-section of FIG. 1 on II-II;

FIG. 3 is a perspective view of a protective eyewear device in accordance with the invention;

FIG. 4 is a plan view of the protective eyewear device of FIG. 3;

FIG. 5 is partial cross-section of FIG. 3 on V-V;

FIG. 6 is schematic cross-section of a protective eyewear device in accordance with the invention showing its laminated structure; and

FIG. 7 is a schematic view of a protective eyewear device according to the invention fitted to a patient.

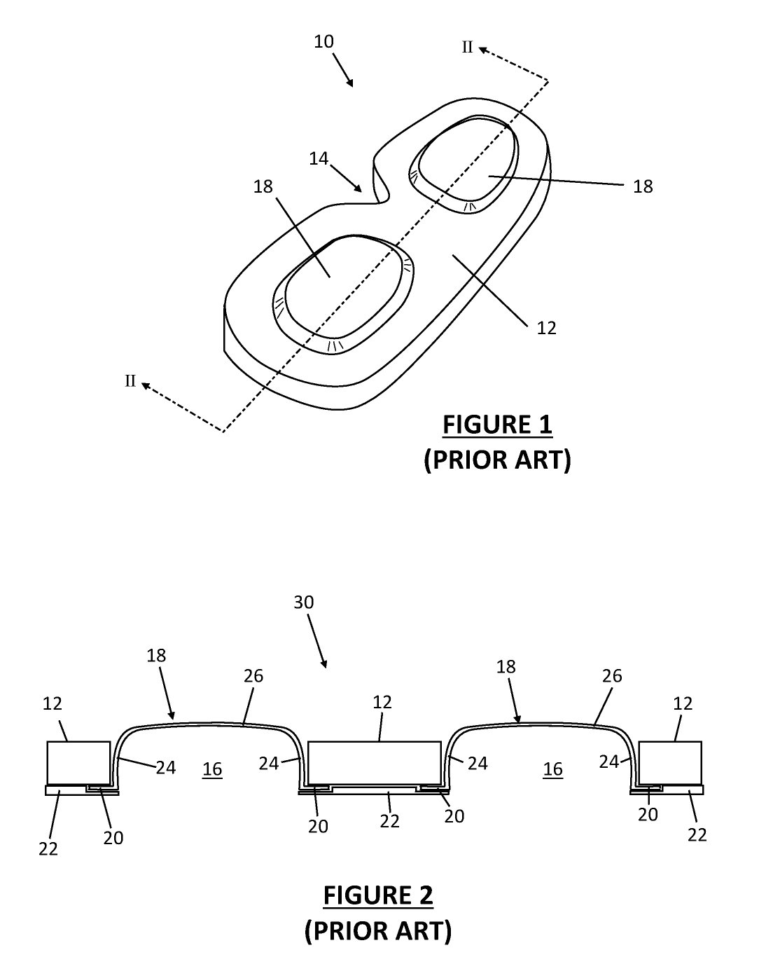

### DETAILED DESCRIPTION

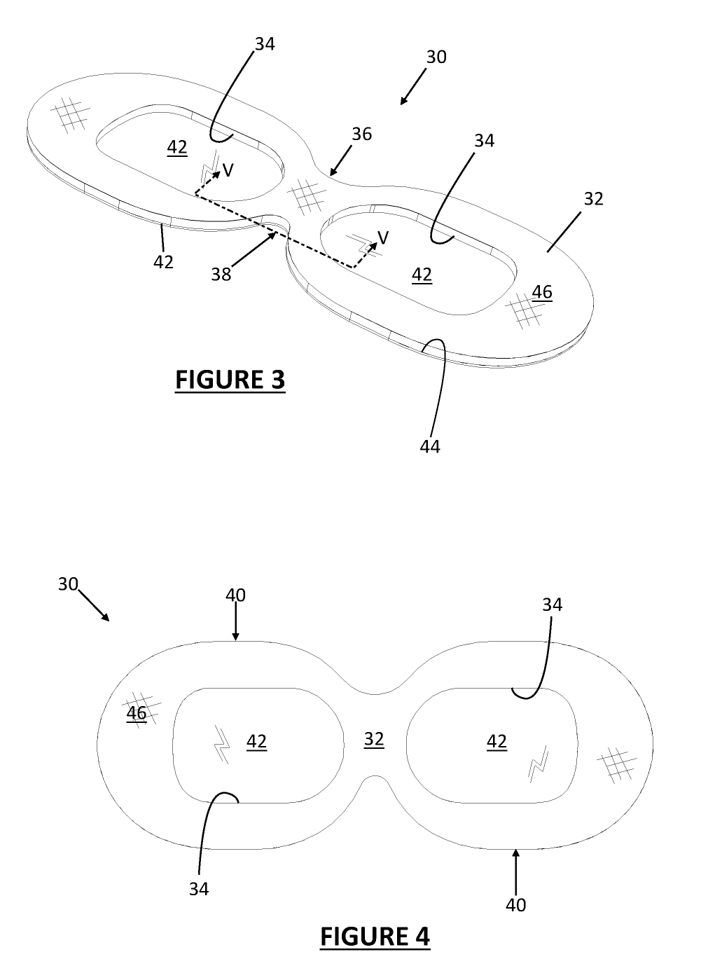

Referring to FIGS. 3, 4 and 5 of the drawings, a protective eyewear device 30 in accordance with the invention comprises a main body 32 manufactured from a foam material (preferably a closed-cell polymer foam). The main body 32 is approximately 10 to 30 mm thick, and may provide shock absorption against something striking it, in use: the force of the impact may be cushioned by the foam layer 32.

The main body 32 comprises a pair of ocular apertures 34 formed as rounded, spaced-apart through holes in the main body 32. The shape, size and spacing of the ocular apertures 34 are selected to correspond to as broad a range of patient populations as possible, but in one embodiment, the ocular apertures are approximately 65 mm wide at their widest point, approximately 43 mm high, and spaced apart by approximately 23 mm. Notably, the increased vertical height of the ocular apertures, compared with known eye protectors, enables the main body portion surrounding the ocular apertures to avoid the patient's eyebrows. This means that when stuck to a patient's face, the connection is generally to skin, rather than to the eyebrows. Typically, the overall width of the protective eyewear device 30 is approximately 215 mm and the overall height of the protective eyewear device is approximately 80 mm.

The main body 32 of the protective eyewear device 30 comprises a pair of nose cut-outs 36, 38 located centrally on opposite long sides 40 of the main body. A first one of the nose cut-outs 38 extends into the main body 32 by approximately 27 mm and is approximately 75 mm wide at its widest point; whereas the second one of the nose cut-outs 36 extends into the main body 32 by approximately 20 mm and is approximately 75 mm wide at its widest point. Thus, the nose cut-outs 36, 38 are shaped to fit different patient populations. It will be appreciated that other dimensions may be used within the scope of the invention.

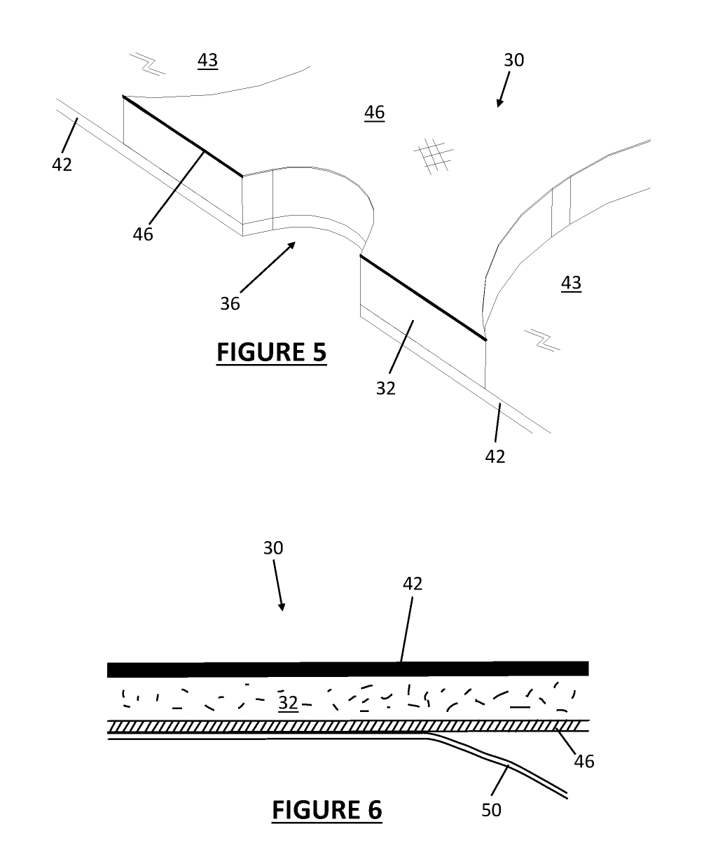

The protective eyewear device 30 further comprises, on one of its flat surfaces, a flat, thin, transparent plastics sheet 42, typically manufactured from clear acetate or acrylic, which is bonded to one entire surface 44 of the main body 32. The plastics sheet 42 completely overlies the ocular apertures 34, thereby forming, as can be seen in FIG. 5 in particular, a hollow cavity 43 overlying, in use, each of the patient's eyes. Such a configuration provides, in use, a barrier between the environment and the wearer 16. Due to the flexibility of the main body 32 and the plastics sheet 42, the protective eyewear device 30 can be bent to confirm to the profile of a patient's face, in use.

A notable, and somewhat surprising, advantage of the invention, is that because the main body 32 and the plastics sheet 42 are flexible and are able to conform to the shape of the patient's face, in use, this causes the plastics sheet 42, in particular, to adopt a curved form in use. The curvature of the plastics sheet 42, in use, serves to 1) tension it slightly; and 2) make it more rigid that would be in its flat state. Thus, even a thin plastics sheet 42 can become quite “rigid”, in use, due to the curvature of the sheet 42 when the protective eyewear device 10 of the invention is stuck to a patient's face. This, surprisingly and advantageously, enables the plastics sheet 42 of the invention to be manufactured from considerably thinner and more flexible materials than the “blisters” of known eyewear protectors, for a given required “rigidity” in use.

Further, in an embodiment of the invention 30, the plastics sheet 42 covers the entire, exterior surface area of the main body portion 32, which means that the protective eyewear device 30 can be easily wiped clean during procedures.

The protective eyewear device 30 is held in-situ by a pressure-sensitive self-adhesive layer 46 that overlies the opposite surface 48 of the main body 32. The self-adhesive layer 46 can be stuck to a patient's face to hold the protective eyewear device 30 in position, in use. The pressure sensitive self-adhesive layer 46 provides a means for temporarily (and ideally sealingly) affixing the protective eyewear device 30 to the wearer's face. The adhesive layer 46 substantially covers the entire inner surface area of the main body portion 32, such that the entire periphery of the device 30 can be (sealingly) affixed to the wearer's face.

A further advantage of the invention being formed from sheet-like materials, such as thin plastics sheet and polymer foam, is that it can be cut by a surgeon, for example very easily, using a pair of conventional scissors. This enables the shape of the eyewear device to be customised in-theatre and/or for it to be cut in half (across the bridge area) to provide independent eye protectors, or an eye protector for just one of the patient's eyes (a benefit in certain types of surgery, such as maxillofacial and ophthalmic procedures). This is possible, in one respect, because the plastics sheet 42 and the self-adhesive layer 46 cover the entire upper and lower surfaces, respectively, of the main body. This means that it does not matter where the device is cut, unlike with known eye protectors in which care must be taken to ensure that the “blisters” are not separated from the main body portion by the cutting operation.

The laminated structure of the protective eyewear device 30 is shown schematically in FIG. 6 of the drawings, from which it can be seen that the protective eyewear device 30 is formed as a laminate comprising the foam main body 32 sandwiched between the pressure-sensitive self-adhesive layer 46 on one side and the plastics sheet 42 on the opposite side. Also shown in FIG. 6 is a peel-off protective layer 50 which covers the self-adhesive layer 46 to protect it and prevents inadvertent sticking prior to use. In this embodiment, the invention may be manufactured in a cost-effective process, namely by laminating layers of foam, adhesive, and cover web. The shapes may be pre-cut or cut separately.

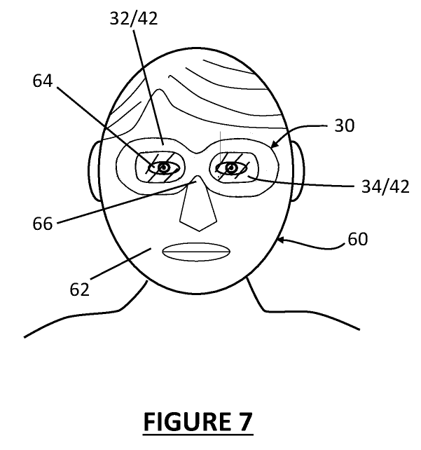

Referring now to FIG. 7, the protective eyewear device 30 is fitted to a patient 60, in use. The protective eyewear device 30 is easily deformable and may thus be bent to conform to the shape of the wearer's face 62. The protective eyewear device 30 extends horizontally to a point laterally beyond the wearers eyes 64, and terminates at or near the wearer's temples, such that the wearer's 64 eyes are entirely covered. The upper and lower horizontal edges 40 of the main body portion 32 each comprise a central nose cut-out 36, 38 configured, in use, to fit around the bridge of the wearer's nose 66. The protective eyewear device 30 is configured such that a user can select which nose cut-out 36, 38 best fits the bridge of the wearer's nose 66 by rotating the main body portion 32 between the first and second positions. A good fit between the bridge of the wearer's nose 66 and the selected nose cut-out 36, 38 helps to prevent slippage of the protective eyewear device 30 and to improve comfort.

In use, after removing the peel-off layer 50 and subsequently exposing the self-adhesive layer 46, the main body portion 32 of the protector 30 can be pressed onto the wearer's face 62, ensuring that the correct nose cut-out 36, 38 has been selected and correctly aligned with the bridge of the wearer's nose 66; and both eyes 64 are entirely covered, and align with the corresponding ocular apertures 34. Then, by applying a small amount of pressure to the exterior surface of the main body 32, the self-adhesive layer 46 provides a seal between the inner surface of the device 30 and the face 62. Once sealingly affixed, the eyewear 30 provides a barrier between the environment and the wearer's eyes 64.

Removal of the eyewear 30, is accomplished by gently peeling it away from the wearer's face. The protective eyewear device 30 is ideally a single-use, disposable item.

In an alternative embodiment (not shown), the protective eyewear device comprises a single elongate ocular aperture, which extends horizontally as a slot across the main body portion 32. In another possible embodiment, each eye is protected separately by separate eye patches formed as annular foam main bodies covered on one side by a web of transparent plastics sheet and having a peripheral self-adhesive layer disposed on the opposite side of the main body to the transparent plastics sheet.

The invention is not restricted to the details of the foregoing embodiments, which are merely exemplary of the invention. For example, the shapes, configurations and materials described are illustrative, rather than limiting.

1. A protective eyewear device comprising:
a deformable main body portion, the main body portion comprising one or more ocular apertures;
a pair of dissimilar cut-outs each intersecting an opposite edge of the main body portion and each being adapted, in use, to conform to dissimilar wearer's nose bridge shapes;
a protective layer comprising a thin, flat sheet of plastics material affixed to a first surface of the main body; and
an adhesive layer affixed to a second surface of the main body portion opposite the first surface.

2. The device of claim 1, wherein the protective layer is continuous and covers the entire surface of the first surface of the main body.

3. (canceled)

4. The device of claim 1, wherein the main body is resiliently deformable and manufactured from a sheet of foam.

5. (canceled)

6. The device of claim 5, wherein the foam comprises as any one or more of the group consisting of: an open-cell polymer foam; a closed-cell polymer foam; an open-cell elastomer foam; a closed-cell elastomer foam; an open-cell memory foam; and a closed-cell memory foam.

7. The device of claim 1, comprising a generally slot-shaped ocular aperture whose periphery is selected to surround both eyes of a patient, in use.

8. The device of claim 1, comprising a pair of spaced-apart ocular apertures, each ocular aperture having a periphery that is shaped and dimensioned to surround an eye of a patient, in use, and which ocular apertures are spaced-apart so as, in use, to register with the patient's eyes.

9. The device of claim 1, wherein the ocular aperture, or apertures, are shaped and sized to correspond with the eyes of the wearer, such that the wearer's eyes are not obstructed, and/or are visible when wearing the protector.

10. (canceled)

11. The device of claim 1, wherein the main body portion has an overall width selected to extend, in use, laterally past the wearer's eyes.

12. The device of claim 1, wherein the adhesive layer comprises a self-adhesive layer.

13. The device of claim 12, wherein the self-adhesive layer comprises a pressure-sensitive, self-adhesive layer.

14. The device of claim 1, wherein the adhesive layer substantially covers the entire inner surface area of the main body portion.

15. (canceled)

16. (canceled)

17. (canceled)

18. (canceled)

19. The device of claim 1, comprising a laminated structure comprising a foam main body sandwiched between a pressure-sensitive self-adhesive layer on one side and a transparent acetate sheet on the other side.

20. (canceled)

21. (canceled)

22. The device of claim 1, wherein the protective layer is translucent or transparent.

23. The device of claim 1, wherein the protective layer is flexible to permit the device to conform, in use, to the shape of a patient's face.

24. The device of claim 1, wherein the protective layer comprises an acetate sheet.

25. The device of claim 13, comprising a peel-off protective layer adapted to over the self-adhesive layer.
